(19) United States
(12) Patent Application Publication
(10) Pub. No.: US 2007/0089362 A1
(43) Pub. Date: Apr. 26, 2007

(54) Method for pine beetle removal from trees

(76) Inventor: Zachary A. Fields, Douglas, TX (US)

Correspondence Address: MEYERTONS, HOOD, KIVLIN, KOWERT & GOETZEL, P.C., 700 LAVACA, SUITE 800, AUSTIN, TX, 78701, US

(21) Appl. No.: 11/254,954

(22) Filed: Oct. 20, 2005

(51) Int. Cl. A01H 3/00

(52) U.S. Cl. 47/58.1 R

(57) ABSTRACT

In some embodiments, a system including a chemical composition and a drill may be used to reduce insect infestation of trees. A chemical composition may include one or more lightweight hydrocarbons. Openings may be formed in a tree. The chemical composition may be added to the openings such that insect infestations of trees may be substantially reduced. In an embodiment, the chemical composition may be added to three or more openings positioned around a circumference of a pine tree such that pine beetles are substantially eradicated from the pine tree.

### BACKGROUND

1. Field of the Invention

The present invention relates to a system and method for the removal of insects from trees. More particularly, the invention relates to the removal of pine beetles from pine trees.

2. Description of Related Art

Insect infestations can spread quickly, destroy forests, and cause severe losses to timber industries. For example, the southern pine beetles and mountain pine beetles are among the most destructive insects to pine trees. From 1999 to 2001 in Tennessee, $358 million of pine timber was lost on approximately 390,000 acres due to the southern pine beetle. Pine beetle outbreaks may occur in wilderness areas, tree farms, parks, subdivisions, and backyards.

Pine beetles attack various kinds of pine trees, such as southern yellow pines (e.g., loblolly, shortleaf, Virginia, pitch, and Table Mountain pines) and eastern white pines. Dying pines with yellow or red needles are indicators of pine beetle infestation. Pitch tubes and/or exit holes are also indications of pine beetle infestation. Pitch tubes are areas of resin that accumulate where adult pine beetles bore into a pine.

Currently, infested trees are typically cut down or left to die. Surrounding trees may be thinned and/or treated with insecticide to inhibit spread of the pine beetle infestation.

### SUMMARY

In various embodiments, insect infestation of trees may be reduced and/or eliminated allowing for survival of an infested tree or trees. Trees near infested trees may be treated to limit and/or inhibit spread of insect infestation. For example, pine beetle infestation may be at least substantially reduced and/or substantially eliminated. A chemical composition may be introduced in openings formed in a tree to substantially reduce and/or eliminate insect infestation of the tree. In some embodiments, the chemical composition may be introduced into openings formed in trees that are near insect infested trees to inhibit spread of the infestation. In some embodiments, a kit may be supplied to treat insect infested trees and/or trees near an insect infestation. The kit may include a container with the chemical composition. The container may contain the chemical composition under pressure and/or may be able to introduce the chemical composition to a tree at as a pressurized stream of fluid. The kit may also include a drill or other device for forming openings in a tree or trees.

Openings may be provided in a tree. Two or more openings may be created or drilled into a tree. In an embodiment, four or more openings may be formed in a tree. In some embodiments, openings may be formed at any desired positioned in the tree. In some embodiments, openings may be formed at a height of 2-5 feet above the ground. In some embodiments, openings may be formed at a height less than 1 foot above the ground. Openings may be approximately evenly positioned around a circumference of a tree. In some embodiments, 3 to 5 openings may be provided in a tree. Openings may extend at an angle into a tree. Openings may extend into a tree from approximately 30 to approximately 50 degrees relative to an axis parallel to the trunk of the tree.

A chemical composition for treating a tree may be applied to openings in the tree. The chemical composition may be WD-40®. The chemical composition may include one or more lightweight hydrocarbons and/or aliphatic hydrocarbons.

### DETAILED DESCRIPTION

In various embodiments, insect infestation of trees may be reduced or eliminated. A reduction and/or elimination of an insect infestation may allow infested trees to survive. A system and method may be used in infested trees and/or in trees near infested trees to inhibit infestation of unaffected trees. In some embodiments, a chemical composition may be introduced in one or more openings formed in trees, such as pine trees. The chemical composition may reduce or substantially eliminate live pine beetle infestation of a tree. The chemical composition may inhibit the spread of pine beetles to a tree treated with the chemical composition. In an embodiment, a hydrocarbon mixture may be applied to one or more of the openings formed in pine trees. Applying the hydrocarbon mixture to one or more openings in a tree may substantially reduce insect infestation of the tree.

A chemical composition used to treat a tree may include a mixture of one or more hydrocarbons. A hydrocarbon, in the context of this application, refers to compounds that contain only carbon and hydrogen. A chemical composition may include one or more aliphatic hydrocarbons. The aliphatic hydrocarbons may include unbranched hydrocarbons, branched hydrocarbons, or mixtures thereof. The chemical composition may also include tertiary alkyl amines, (e.g., triethyl amine). The tertiary alkyl amine may have a low surface tension. The chemical composition may also include one or more ethers, esters, ketones, alcohols (e.g., octonol), and/or organic acids, (e.g., heptanoic acid). In an embodiment, the chemical composition may include metal ion chelating agents.

In one embodiment, a chemical composition may include a mixture of aliphatic petroleum distillates, petroleum base oil, light hydrocarbons, and/or carbon dioxide. The chemical composition may include approximately 40-60% aliphatic petroleum distillates. In one embodiment, the aliphatic petroleum distillates may be composed of a refined petroleum distillate that boils in the range of about 149° C. (300° F.) to about 205° C. (400° F.). In one embodiment, the aliphatic petroleum distillates may be Stoddard solvent. The chemical composition may also include approximately 10-30% of petroleum base oil. In one embodiment, petroleum base oil may include a mixture of hydrocarbons that have been obtained by the removal of normal paraffins from a petroleum fraction by solvent crystallization. In an embodiment, the petroleum base oil includes hydrocarbons having a carbon number predominantly in the range of C20 through C50. The chemical composition may also include light hydrocarbons. Light hydrocarbons may include hydrocarbons having a carbon number predominantly in the range of C9 through C16. The light hydrocarbons may have a boiling range from about 150° C. (300° F.) to about 290° C. (555° F.). Light hydrocarbons may be obtained by treating a petroleum fraction with hydrogen in the presence of a catalyst.

In some embodiments, the chemical composition may include approximately 1-4% carbon dioxide or other inert gases (e.g., nitrogen, helium, argon, etc.). Carbon dioxide or inert gases may act as a propellant to allow the hydrocarbons to be applied to the tree as a spray. Examples of commercially available chemical compositions that maybe used to treat pine tress include, but are not limited to, Rus-Kil Lube 60 available from (BLP Mobile Paints, Theodore AL), United 139 lubricant (available from United Laboratories, St. Charles, Ill.), Mr. Mechanic Lubricant (available from Quest Chemical Company, Houston, TX), WD-40®(available from the WD-40 Company, San Diego, Calif.), Performance HF Lube 77 (available from Performance Chemicals LTD., United Kingdom), and RD-90 Spray Lubricant (available from Sprayway Inc., Addison, Ill).

The chemical composition may be capable of lubrication at low temperatures. The chemical composition may have a lower viscosity than lightweight machine oil (34.2 centipoise (cp)). The chemical composition may have a viscosity less than approximately 1 cp. The chemical composition may have a low surface tension. The chemical composition may be insoluble in water. The chemical composition may have a boiling point of approximately 320-350 ° F. The chemical composition may have a vapor pressure of approximately 105 to 112 psi at 70° F. The chemical composition may include between approximately 50 and 80% volatile compounds. The chemical composition may have a vapor density greater than air. The chemical composition may have a specific gravity of approximately 0.8 to 0.825 at 72° F. The chemical composition may be flammable. The chemical composition may have a flash point of approximately 125 to 135° F.

The chemical composition used to treat trees may be a liquid. In some embodiments, the chemical composition may be sprayable. For example, the chemical composition may be in a canister, an aerosol container, or spray bottle. The chemical composition may be injected by a syringe and/or dropped from a pipette into an opening in a tree. The chemical composition may be poured into an opening in a tree.

In some embodiments, one or more openings may be formed in a tree. Two, three, four, five, or more openings may be provided in a tree. In an embodiment, approximately 3 to 5 openings may be formed in a tree. The number of openings provided in a tree may depend on the circumference of the tree. For example, a tree with a narrow trunk may require only 3 openings while a tree with a larger trunk may require 5 openings. Openings may be positioned around a circumference of a tree. In an embodiment, the openings may be approximately evenly positioned around the circumference of the tree.

Openings may have various cross-sectional shapes, such as substantially rectangular, substantially square, substantially oval, or irregular. An opening may have a substantially circular cross-sectional shape. Openings in a tree may have a diameter or cross-sectional width of less than approximately 1 inch. The openings may have a diameter or cross-sectional width of less than approximately ½ inch. The openings may have a diameter or cross-sectional width of approximately 3/16 to approximately ⅜ inch.

In some embodiments, openings may be formed in a tree by drilling into the tree. Drilling openings in a tree may include using a drill, such as a power drill, tree borer, and/or manually operable drill. In some embodiments, openings may be formed in a tree by driving, vibrating, rotating, and/or impacting a device (e.g., a punch, awl, nail, screw or blade) into the tree. After insertion, the device used to form the opening may be removed. Openings may be formed at any height in the trunk or limb of a tree to be treated. In some embodiments, openings may be formed in the tree at a height less than 6 feet above the ground. In some embodiments, openings may be formed at a height of less than 2 feet above the ground. In some embodiments, openings may be formed in the approximately 3 to 6 inches from the ground. In some embodiments, two or more rings of openings may be formed in the tree. The openings in a first ring may be offset from the openings in a second ring so that one or more of the first ring openings are not positioned vertically in line with the openings of the second ring.

In some embodiments, openings formed in the tree may extend in the tree to a depth in a range from about 0.5 inches to about 10 inches. In some embodiments, openings may extend into a tree less than 8 inches. In some embodiments, openings may extend into a tree approximately 3 to 4 inches. The openings may extend into a tree at an angle. The openings may extend into a tree at from about 0° to about 85° relative to an axis parallel to a tree trunk. In an embodiment, the openings may extend into a tree at an angle greater than approximately 30° and less than approximately 50° relative to an axis parallel to a tree trunk. The openings may extend into a tree at approximately 45° relative to an axis parallel to the tree trunk. Opening in the same tree may extend in the tree at substantially the same angle, or the openings may be formed at different angles.

In some embodiments, the chemical composition may be applied to the openings formed in a tree. The chemical composition may be applied or introduced into the openings as a pressurized fluid. Openings in the tree may be at least partially filled with the chemical composition. In an embodiment, openings in the tree may be substantially filled with the chemical composition. For example, an aerosol container including the chemical composition may be sprayed into the openings in the tree.

Applying the chemical composition to openings in trees may substantially reduced insect infestation of trees. In some embodiments, a pine beetle infestation may be substantially eliminated from a tree in less than a week by applying the chemical composition to openings in the tree. Tree loss due to insect infestation, such as pine beetles infestation, may be substantially reduced and/or eliminated by applying the chemical composition to openings in trees.

In some embodiments, a kit may be provided for insect infestation reduction. A kit may include a container including the chemical composition and a drill or other device for forming openings in a tree or trees to be treated. The container may be a spray bottle or an aerosol container. The container may include a tube or funnel to facilitate application of the chemical composition to the openings in the trees. An operator may use the kit to form openings in one or more trees and apply the chemical composition to the openings in the trees.

In some embodiments, the chemical composition may be introduced into openings in a tree as a pressurized stream of fluid and/or gas. A container may contain the chemical composition under pressure. In an embodiment, the container may be any pressurized device or container capable of maintaining the chemical composition under a pressure greater than atmospheric pressure. The container may include a device capable of delivering the chemical composition as a pressurized fluid stream. For example, the container may include a nozzle capable of delivering the chemical composition as a pressurized fluid stream. When the chemical composition is released from the container, the chemical composition may be delivered as a pressurized stream of fluid. The container may be capable of introducing the chemical composition in openings in a tree at a pressure greater than atmospheric pressure. In an embodiment, the container may be capable of introducing the chemical composition in openings in a tree at a pressure greater than 1 atm.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method of removing pine beetles from pine trees comprising:
forming one or more openings in a pine tree; and
introducing a chemical composition to one or more of the openings in the pine tree, wherein the chemical composition comprises one or more hydrocarbons;
wherein introducing the chemical composition to one or more of the openings substantially reduces the number of live pine beetles in the pine tree.

2. The method of claim 1, further comprising forming two or more openings in the pine tree.

3. The method of claim 2, wherein the openings are positioned around a circumference of the pine tree.

4. The method of claim 2, wherein the openings are approximately evenly positioned around a circumference of the pine tree.

5. The method of claim 1, further comprising forming four or more openings in the pine tree.

6. The method of claim 1, wherein forming one or more openings comprises drilling openings in the pine trees.

7. The method of claim 1, wherein at least one of the openings extend into the pine tree at an angle from approximately 0 degrees to approximately 85 degrees relative to an axis parallel to a trunk of the pine tree.

8. The method of claim 1, wherein at least one of the openings extend into the pine tree at an angle from approximately 30 degrees to approximately 50 degrees relative to an axis parallel to a trunk of the pine tree.

9. The method of claim 1, wherein one of the openings has a diameter of approximately 3/16 inch to approximately ⅜ inch.

10. The method of claim 1, wherein one or more of the openings are positioned at a height approximately within 6 feet of the ground.

11. The method of claim 1, wherein the chemical composition comprises one or more aliphatic hydrocarbons.

12. The method of claim 1, wherein the chemical composition comprises aliphatic petroleum distillates.

13. The method of claim 1, wherein the chemical composition comprises petroleum base oil.

14. The method of claim 1, wherein the chemical composition comprises light hydrocarbons, wherein the light hydrocarbons have a carbon number from 9 to 16.

15. The method of claim 1, wherein the chemical composition comprises:
aliphatic petroleum distillates;
petroleum base oil; and
light hydrocarbons.

16. The method of claim 1, wherein the chemical composition comprises WD-40®.

17. The method of claim 1, wherein the chemical composition is deliverable by an aerosol can.

18. The method of claim 1, wherein a boiling point of the chemical composition is approximately 320-350° F.

19. The method of claim 1, wherein a viscosity of the chemical composition is less than approximately 1 cps.

20. The method of claim 1, wherein a specific gravity of the chemical composition is approximately 0.8 to 0.825 at 72° F.

21. The method of claim 1, wherein introducing the chemical composition to one or more of the openings in the pine tree comprises delivering the chemical composition as a pressurized stream of fluid.

22. A pine beetle removal kit comprising:
an aerosol can comprising a chemical composition, wherein the chemical composition comprises one or more hydrocarbons; and
a drill.

23. A method of removing insects from trees comprising:
forming one or more openings in a tree; and
introducing a chemical composition to one or more of the openings in the tree wherein the chemical composition comprises one or more hydrocarbons;
wherein introducing the chemical composition to one or more of the openings substantially reduces the number of live insects in the tree.
